(19) United States
(12) Patent Application Publication
(10) Pub. No.: US 2024/0385170 A1
(43) Pub. Date: Nov. 21, 2024

(54) SYSTEMS AND METHODS FOR AUTOMATED LIGHT SOURCE MODULATION FOR TESTING OF A PHOTOPLETHYSMOGRAM SENSOR

(71) Applicant: PLUME DESIGN, INC., Palo Alto, CA (US)

(72) Inventors: Min-Feng HSIEH, Tainan City (TW); Ta-Lung LEE, New Taipei City (TW); Shu-Hua CHANG, Zhubei City (TW); Zhicheng QIU, Cupertino, CA (US)

(21) Appl. No.: 18/318,065

(22) Filed: May 16, 2023

(51) Int. Cl. G01N 33/49

(52) U.S. Cl. CPC G01N 33/4925; A61B 5/02416; A61B 2560/0223

(57) ABSTRACT

Systems and methods of embodiments of the present disclosure provide automated testing of PPG sensors using a programmatically controlled light source. The light source may include one or more light emitting elements that can be modulated to emit light towards the PPG sensor to simulate reflectivity of the tissue of a subject. Particular heart rates, heart rate variabilities and/or other physiological behaviors may be simulated based on illumination patterns, including, e.g., frequency, range of intensity, or variations thereof, among other illumination pattern characteristics. Based on the output signal produced by the PPG sensor in response to the illumination pattern, the PPG sensor may be assessed for accuracy and/or sensitivity to ensure quality.

### FIELD OF TECHNOLOGY

The present disclosure generally relates to an automated light source and systems and methods of modulation thereof for automated testing of a photoplethysmogram (PPG) sensor.

### BACKGROUND OF TECHNOLOGY

A PPG is an optically obtained plethysmogram that can be used to detect blood volume changes in the microvascular bed of tissue. A PPG sensor may detect a time varying light absorbance signal (“PPG” signal) in order to detect time-varying blood volume changes to, e.g., measure heart rate and/or heart rate variability. The time varying PPG signal may be affected by a number of factors, some of which include the optical properties of the tissues and blood at the measurement site, volume of arteries near the skin's surface and the wavelength of the light source.

### SUMMARY OF DESCRIBED SUBJECT MATTER

In some embodiments, PPG sensors may measure the time varying PPG sensor based on reflected light. Thus, a PPG sensor may emit light onto the skin of a subject. While much of the light may be absorbed by the skin and tissue of the subject, at least a portion may be reflected back to the PPG sensor. A photo-detector (PD) may detect the reflected light, and as light varies through time, the blood volume changes in the tissue may be determined to determine physiological measurements such as heart rate and heart rate variability, among others.

Because the PPG sensor relies on reflected light from the tissue of the subject, the PD may be designed to operate at a sensitivity level sufficient to detect variations in the reflected light. But not all PDs may be of sufficient quality for such detection. Thus, each PD and/or a sample of PDs may be tested to ensure quality. But testing each PD on a human may be impractical to testing large numbers of PDs due to time and personnel/test subject constraints, as well as an inability to control with precision the physiological measurement of each human to ensure accuracy of each PD.

Thus, embodiments of the present disclosure provide automated testing of PPG sensors using a programmatically controlled light source, such as a lamp or a light emitting diode (LED). The light source may include light emitting elements that can be controlled to project light towards the PPG sensor to simulate reflectivity of the tissue of a subject. Particular heart rates, heart rate variabilities and/or other physiological behaviors may be simulated based on illumination patterns, including, e.g., frequency, range of actuation, or variations thereof, among other illumination pattern characteristics. Based on the physiological measurement output by the PD in response to the illumination pattern, the PPG sensor may be assessed for accuracy and/or sensitivity to ensure quality.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 depicts a testing fixture for automated testing and calibration of PPG sensor devices in accordance with one or more embodiments of the present disclosure.

FIG. 2 depicts a light source controller of a testing fixture for automated testing of PPG sensor devices using a light source in accordance with one or more embodiments of the present disclosure.

FIG. 3 depicts a calibration engine of a testing fixture for automated calibration of PPG sensor devices based on testing with a light source in accordance with one or more embodiments of the present disclosure.

FIG. 4 depicts a processing system for a testing fixture for automated testing and calibration of PPG sensor devices in accordance with one or more embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

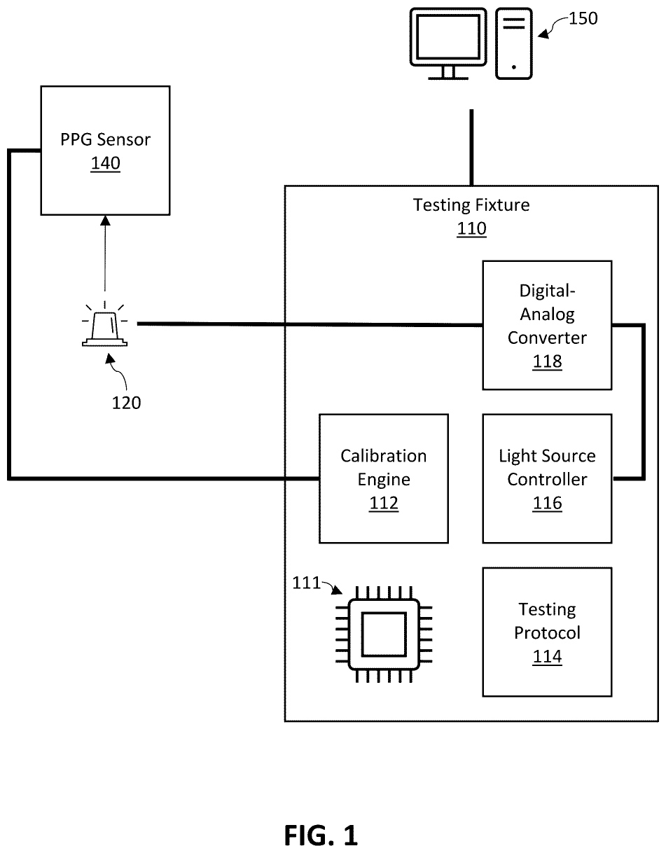

FIGS. 1 through 4 illustrate systems and methods of automated PPG sensor testing for calibration and/or validation of devices employing a PPG sensor, such as wearable devices (e.g., smartwatches, smart rings, smart bands, etc.), medical PPG devices, among others or any combination thereof. The following embodiments provide technical solutions and technical improvements that overcome technical problems, drawbacks and/or deficiencies in the technical fields involving PPG measurement and analysis (e.g., physiological measurement monitoring for medical and/or consumer devices). Such technical problems may involve variability and/or inconsistency of PGG sensor sensitivity and accuracy when incorporated into devices. Such PPG sensors may have some degree of inconsistency from one sensor to another due to manufacturing tolerances and other factors.

As explained in more detail, below, technical solutions and technical improvements herein include aspects of improved testing equipment that enable automated, controlled and consistent testing of PPG sensors and/or devices incorporating PPG sensors. To ensure that a device may operate effectively, e.g., with a threshold degree of accuracy between an output signal and an actual condition of a subject, the PPG sensor may be tested using simulated physiology. The simulated physiology, e.g., via a controllable light source, enables a testing fixture to controllably define the physiological behavior to be measured and assess the accuracy of the PPG sensor in measuring the physiological behavior. Based on the accuracy, the PPG sensor may be calibrated for improved accuracy and/or discarded as defective. Such technical solutions enable efficient, scalable testing and/or calibration for improved device accuracy.

Based on such technical features, further technical benefits become available to users and operators of these systems and methods. Moreover, various practical applications of the disclosed technology are also described, which provide further practical benefits to users and operators that are also new and useful improvements in the art.

Referring now to FIG. 1, a testing fixture for automated testing and calibration of PPG sensor devices is illustrated in accordance with one or more embodiments of the present disclosure.

In some embodiments, a testing fixture 110 may power a light source 120 to modulate an intensity of illumination according to a testing protocol 114. In some embodiments, the testing fixture 110 may include one or more software and/or hardware components for performing automated testing of a PPG sensor 140 based on a sensor testing parameters selected and/or input by a user via a user computing device 150. The sensor testing parameters may be defined according to a particular simulated physiological behavior for which the PPG sensor 140 is to be tested.

Accordingly, in some embodiments, the testing fixture 110 may use the sensor testing parameters to identify and/or generate a testing protocol 114 defining an illumination pattern. Based on the illumination pattern, in some embodiments, the testing fixture 110 may implement an light source controller 116 to generate control signals configured to cause the light source 120 to illuminate according to the illumination pattern. The testing fixture 110 may also include a digital-to-analog (DA) converter 118 to provide analog control signals to control the light source 120. In some embodiments, the PPG sensor 140 may continuously or periodically measure reflectivity and/or reflectivity variations as simulated by the illumination intensity of the light source 120 to output time-varying PPG sensor data. In some embodiments, the testing fixture 110 may employ a calibration engine 112 to receive time-varying PPG sensor data from the PPG sensor 140 in response to the modulation of illumination intensity of the light source 120 to determine an error and/or to calibrate the PPG sensor 140.

In some embodiments, the testing fixture 110 may include hardware components such as a processor 111, which may include local or remote processing components. In some embodiments, the processor 111 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor 111 may include data-processing capacity provided by the microprocessor. In some embodiments, the microprocessor may include memory, processing, interface resources, controllers, and counters. In some embodiments, the microprocessor may also include one or more programs stored in memory.

Similarly, the testing fixture 110 may include storage, such as one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the storage may include, e.g., a suitable non-transient computer readable medium such as, e.g., random access memory (RAM), read only memory (ROM), one or more buffers and/or caches, among other memory devices or any combination thereof.

In some embodiments, the testing fixture 110 may employ computer engines to implement the calibration engine 112, the light source controller 116, DA converter 118 and/or the testing protocol 114. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, the calibration engine 112, the light source controller 116, DA converter 118 and/or the testing protocol 114 may include dedicated and/or shared software components, hardware components, or a combination thereof. For example, the calibration engine 112, the light source controller 116, DA converter 118 and/or the testing protocol 114 may each include a dedicated processor and storage. However, in some embodiments, one or more of the calibration engine 112, the light source controller 116, DA converter 118 and/or the testing protocol 114 may share hardware resources, including the processor 111 and storage of the testing fixture 110 via, e.g., a bus.

In some embodiments, the light source 120 may include an electrically driven light source (e.g., electrical energy discharge). For example, the light source 120 may include one or more, e.g., light emitting diodes (LEDs), organic LEDs (OLEDs), light-emitting electrochemical cells, electroluminescent wires, field-induced polymer electroluminescents, lasers, arc lamps, electrodeless lamps, excimer lamps, fluorescent lamps, high intensity discharge lamps, hollow cathode lamps, induction lighting, neon and/or argon lamps, plasma lamps, xenon flash lamps, or among others or any combination thereof.

In some embodiments, the light source 120 may include one or more LEDs configured to emit one or more predefined wavelengths of light. In some embodiments, the LED(s) may be selected based on a wavelength that the PPG sensor 140 is designed and/or optimized to detect. For example, some PPG sensors utilize photodetectors optimized for one or more particular wavelengths of light, such as, green, red, infrared (IR), ultraviolet (UV) or other portion of the electromagnetic spectrum or any combination thereof. Indeed, as further detailed below, the photodetector may be paired with one or more LEDs of the PPG sensor 140 that are configured to emit the one or more portions of the electromagnetic spectrum that the photodetector is optimized to detect. Thus, the light source 120 may similarly be one or more LEDs configured to emit light of the portion(s) of the electromagnetic spectrum that the photodetector is optimized to detect, such as, e.g., a combination of one or more green LEDs, red LEDs, IR LEDs and/or UV LEDs. In some variations, the light emitting diodes of the light source 120 may emit light with a peak spectral response between about 400 and 620 nm.

In some embodiments, to simulate reflectivity of tissue of the subject during a cardiac behavior, the light source 120 may configured to emit light that approximates the portion(s) of the electromagnetic spectrum and intensity of light emitted by the PPG sensor 140 and reflected off the tissue. Such light reflected off the tissue varies in intensity as blood flow through the tissue varies due to stages in the cardiac cycle, and therefore being indicative of the cardiac behavior. Thus, the light source 120 may be controlled to vary the light emitted therefrom to simulate the variation in light reflected by the tissue during the cardiac behavior.

In some embodiments, the light source 120 may operate by converting electrical energy into radiation, e.g., in the form of light from the one or more portions of the electromagnetic spectrum. Thus, in some embodiments, to simulate the light reflected by the tissue during the cardiac behavior, the testing fixture 110 may produce a control signal that modulates the light source 120 to vary in intensity, frequency and/or wavelength by controlling each of the one or more LEDs to emit light that varies according to the variation in light reflected by the tissue as per the cardiac behavior. For example, the light source 120 may employ a red LED, and the intensity of the red LED may be varied according to an expected variation in red light reflected by the tissue of the subject due to blood flow variations resulting from the cardiac behavior. Similarly, the light source 120 may include other LEDs for one or more other portions of the electromagnetic spectrum, either instead of or in combination with a red LED, that may be varied according to the expected variation of each respective portion of the electromagnetic spectrum.

In some embodiments, the light source 120 may be powered and modulated with a control signal to cause the light source 120 to emit light at a frequency, wavelength and/or intensity based on the voltage and/or current of the control signal. As a result, the light source 120 may be controlled to emit a particular illumination characterized by a particular intensity, wavelength and/or frequency to the PPG sensor 140. In some embodiments, the light source 120 may successively present a series of variations to the illumination to the PPG sensor 140 based on a testing protocol. The testing protocol 114 may define an expected output signal from the PPG sensor 140 so as to evaluate the accuracy of the PPG sensor 140 in producing an output signal that matches the expected output signal.

In some embodiments, the testing protocol 114 may include an illumination pattern that is design to match the expected output signal. For example, the testing protocol 114 may be a “normal heart rate” testing protocol that includes testing parameters corresponding to the blood flow and/or heart rate of a 60 beats per minute heart rate. Thus, the illumination pattern may establish a series of intensity levels that would be expected to be measured as the 60 beats per minute heart rate, e.g., by presenting one or more particular intensity levels 60 times per minute for a particular amount time period each time in order to simulate the one or more stages in the cardiac cycle. For example, a relative higher intensity level may be associated with a ventricular ejection stage of the cardiac cycle, whereas a relative lower intensity level may be associated with a ventricular filling stage based on the effects of each stage on blood flow, with or without one or more additional intermediate intensity levels.

Accordingly, in some embodiments, a user may define a testing protocol to test the accuracy of the PPG sensor 140 in producing an output signal that matches a particular expected output signal. The user may interface with a graphical user interface (GUI) of the user computing device 150 to select one or more sensor testing parameters, such as, e.g., a cardiac-related condition to simulate and test (e.g., a heart rate, heart rate variability, heart rate pattern such as a heart rate pattern indicative of, e.g., angina, atrial fibrillation, ventricular fibrillation, among others), or an artificial heart rate pattern, such as a sequence of multiple intensity levels for a specified period of time each not corresponding to a natural heart rate pattern. In some embodiments, the sensor testing parameters may define a custom testing protocol 114 with user specified cardiac behaviors, such as, e.g., heart rate, heart rate variability, and other characteristics of cardiac behavior. In some embodiments, the sensor testing parameters may include, e.g., a number of intensity levels, a frequency of switching between intensity levels, a duration of presentation of each intensity level, a number of times to switch between intensity levels, an amount of time for the testing protocol, among other sensor testing parameters or any combination thereof.

In some embodiments, the testing fixture 110 may receive, from the computing device 150, the sensor testing parameters defined by the user. Therefore, in some embodiments, the testing fixture 110 be in communication with the user computing device 150 via one or more computer interfaces. In some embodiments, one or more computing interfaces may utilize one or more software computing interface technologies, such as, e.g., Common Object Request Broker Architecture (CORBA), an application programming interface (API) and/or application binary interface (ABI), among others or any combination thereof. In some embodiments, an API and/or ABI defines the kinds of calls or requests that can be made, how to make the calls, the data formats that should be used, the conventions to follow, among other requirements and constraints. An “application programming interface” or “API” can be entirely custom, specific to a component, or designed based on an industry-standard to ensure interoperability to enable modular programming through information hiding, allowing users to use the interface independently of the implementation. In some embodiments, CORBA may normalize the method-call semantics between application objects residing either in the same address-space (application) or in remote address-spaces (same host, or remote host on a network).

In some embodiments, one or more computing interfaces may utilize one or more hardware computing interface technologies, such as, e.g., Universal Serial Bus (USB), IEEE 1394 (Fire Wire), Ethernet, Thunderbolt™, Serial ATA (SATA) (including eSATA, SATAe, SATAp, etc.), among others or any suitable combination thereof.

In some embodiments, the testing fixture 110 be in communication with the user computing device 150 via one or more network connections. In some embodiments, the network may include any suitable computer network, including, two or more computers that are connected with one another for the purpose of communicating data electronically. In some embodiments, the network may include a suitable network type, such as, e.g., a public switched telephone network (PTSN), an integrated services digital network (ISDN), a private branch exchange (PBX), a wireless and/or cellular telephone network, a computer network including a local-area network (LAN), a wide-area network (WAN) or other suitable computer network, or any other suitable network or any combination thereof. In some embodiments, a LAN may connect computers and peripheral devices in a physical area by means of links (wires, Ethernet cables, fiber optics, wireless such as Wi-Fi, etc.) that transmit data. In some embodiments, a LAN may include two or more personal computers, printers, and high-capacity disk-storage devices, file servers, or other devices or any combination thereof. LAN operating system software, which interprets input and instructs networked devices, may enable communication between devices to: share the printers and storage equipment, simultaneously access centrally located processors, data, or programs (instruction sets), and other functionalities. Devices on a LAN may also access other LANs or connect to one or more WANs. In some embodiments, a WAN may connect computers and smaller networks to larger networks over greater geographic areas. A WAN may link the computers by means of cables, optical fibers, or satellites, cellular data networks, or other wide-area connection means. In some embodiments, an example of a WAN may include the Internet.

In some embodiments, the user computing device 150 may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

In some embodiments, the testing fixture 110 may determine a testing protocol associated with the sensor testing parameters. For example, the sensor testing parameters may be associated with a particular testing protocol 114 stored in the storage of the testing fixture 110, such as, e.g., where the user specifies a particular cardiac condition. In some embodiments, the sensor testing parameters may define a custom testing protocol 114 with user specified cardiac behaviors, such as, e.g., heart rate, heart rate variability, and other characteristics of cardiac behavior. In some embodiments, store the testing protocol 114 in the storage. In such a case, the testing fixture 110 may generate the testing protocol 114 based on the custom sensor testing parameters.

In some embodiments, the light source controller 116 may access the testing protocol 114 to generate a control signal configured to cause the light source 120 to emit light to present a series of intensity levels to the PPG sensor 140 based on the sensor testing parameters. To do so, in some embodiments, the light source controller 116 may extract the cardiac behavior characteristics from the testing protocol, including, e.g., frequencies and/or durations of each stage in the cardiac cycle. For example, the testing protocol 114 may include the expected output signal, including peaks and troughs in a time-varying signal. The light source controller 116 may correlate the peaks of the time-varying signal to high illumination intensity periods, and the troughs to low illumination intensity periods, and/or anything in between a peak and a trough.

In some embodiments, the time-varying signal may be sampled with a particular resolution, such as a particular, e.g., bit depth. The resolution may define a number of discrete values that can be represented over the range of the magnitudes of the time-varying signal. Thus, the light source controller 116 may sample a period within the time-varying signal of the testing protocol 114 and determine a magnitude, e.g., based on a mean and/or median magnitude within the sample period. The magnitude may be rounded to a nearest value of the set of discrete values to create a single value for that sample period. In some embodiments, the resolution, and thus the set of discrete values, may be configured to be equal to or less than the number of intensity levels achievable by the light source 120 to ensure that all levels in the set of discrete values can be represented with a corresponding intensity level. For example, the light source 120 may be controllable between one or more discrete intensity levels, such as one or more steps in electrical power, or may be continuously variable. In another example, the light source 120 may be controllable between an on state and an off state such that the resolution may be defines with a 2 bit depth corresponding to on or off.

In some embodiments, the testing protocol 114 may use a sampled representation rather than a time-varying signal to represent the sensor testing parameters. As a result, the light source controller 116 may omit the step for sampling the testing protocol 114. For example, the testing protocol 114 may include a bit depth of two such that the testing protocol 114 may be represented as a sequence of binary levels, high and low or on and off.

In some embodiments, the light source controller 116 may have an index of intensity levels to light source 120 positions. Thus, the light source controller 116 may translate the levels in the set of discrete values that represent the testing protocol 114 to the corresponding intensity level. Thus, the sampled representation may be converted or translated to a sequence of intensity levels. In some embodiments, the light source controller 116 may then map the intensity levels in the sequence to voltage and/or current to the light source 120 to achieve each intensity level so as to define an illumination pattern.

In some embodiments, the testing of the PPG sensor 140 may be configured to test the ability of the PPG sensor 140 to detect the highest and lowest intensity level expected in a subject. Thus, in some embodiments, to improve efficiency, the testing protocol 114 may be a sequence of peaks and troughs according to a particular frequency and/or duration of each peak and/or trough.

In some embodiments, the testing protocol 114 may be the illumination pattern and/or the sequence of intensity levels rather than an expected output signal. Thus, the testing protocol 114 may be selected from a library or configured based on the user selections at the user computing device 150. As a result, the light source controller 116 may omit the sampling, translation and mapping steps.

In some embodiments, the light source controller 116 may output the illumination pattern as a digital signal. In some embodiments, to convert the digital signal to a control signal that operates the light source 120, the DA converter 118 may convert the digital signal to an analog signal. In some embodiments, the DA converter 118 may convert an abstract finite-precision number (such as a fixed-point binary number) into a physical quantity (e.g., a voltage or a pressure) to convert finite-precision time series data of the digital signal of the illumination pattern to a continually varying physical signal.

In some embodiments, the DA converter 118 may include, e.g., a pulse-width modulator (PWM), an oversampling DA converter, an interpolating DA converter, a binary-weighted DA converter, a cyclic DA converter, a thermometer-coded DA converter, a hybrid DA converter, among other DA converters or any combination thereof.

In some embodiments, the digital signal may not provide enough power to drive the light source 120. Thus, in addition to converting the digital signal to an analog signal, the DA converter 118 may include an amplifier. In some embodiments, amplifier may be a separate piece of equipment or an electrical circuit within the DA converter 118. Indeed, the amplifier may be a separate piece of equipment from the testing fixture 110 or an electrical circuit within the testing fixture 110. In some embodiments, the amplifier may increase the power of the analog signal to a level sufficient to drive the light source 120.

In some embodiments, the DA converter 118 may provide the analog signal of the illumination pattern to the light source 120. In some embodiments, the DA converter 118 and/or the light source controller 116 may include an index that maps voltage and/or current (e.g., as a steady state electrical signal or via modulation such as pulse width modulate) to intensity levels. Thus, based on the position reported by the position encoder, the DA converter 118 and/or the light source controller 116 may emit control signals for each intensity level in the illumination pattern so as to cause the light source 120 to emit light at a next intensity level in the sequence.

In some embodiments, the DA converter 118 and/or the light source controller 116 may control the light source 120 to simulate a continuous signal using a continuously variable electrical signal, e.g., by scaling current and/or voltage corresponding to the intensity levels of the illumination pattern. To do so, the DA converter 118 and/or the light source controller 116 may modulate the current and/or voltage such that each intensity level in the illumination pattern is produced according to the correct duration according to the illumination pattern. Such a control scheme may also be applied to a light source 120 having discrete steps in intensity throughout a range of intensity levels.

In some embodiments, the illumination pattern may include two intensity levels. Thus, the DA converter 118 and/or the light source controller 116 may control the light source 120 to modulate between the two intensity levels on the light source 120.

In some embodiments, the illumination of the light source 120 may radiate light towards the PPG sensor 140 according to the sequence of intensity levels of the illumination pattern. Accordingly, the PPG sensor 140 may detect the light and output time-varying PPG sensor data that measures the time-varying intensity levels that simulates the cardiac behavior. As result, in some embodiments, the performance of the PPG sensor 140 may be assessed based on the degree to which the time-varying PPG sensor data matches the expected output signal.

In some embodiments, to do so, the calibration engine 112 of the testing fixture 110 may receive the time-varying PPG sensor data and compare the time-varying PPG sensor data to the expected output signal and/or the illumination pattern. In some embodiments, to enable the comparison between the output signal and the expected output signal/illumination pattern, the output signal may be synchronized with the expected output signal/illumination pattern to align the data in time. Thus, the intensity measured by the PPG sensor 140 at any given time may be compared to the intensity defined by the illumination pattern at the same given time.

In some embodiments, the calibration engine 112 may calculate an error based on a deviation between the time-varying PPG sensor data and the expected output signal and/or the illumination pattern. In some embodiments, the calibration engine 112 may measure an error including a deviation through time between the intensity detected by the PPG sensor 140 according to the time-varying PPG sensor data and the intensity at a same time of the expected output signal and/or the illumination pattern. Based on the deviation through time, the calibration engine 112 may generate an error metric indicative of the deviation through time, such as, e.g., by calculating an average, median, or other statistical metric of the deviation through time.

In some embodiments, the calibration engine 112 may calculate the error as a number or frequency of mismatches between the output signal and the expected output signal/illumination pattern. A mismatch may include one intensity level measured by the PPG sensor 140 and a different intensity level define in the illumination pattern for the same time.

In some embodiments, where the error, including the deviation and/or the number of mismatches, exceeds a predetermined threshold, the calibration engine 112 may determine that the PPG sensor 140 is defective. In some embodiments, the predetermined threshold may be a measure relative to a baseline. For example, for a deviation in amplitude, the predetermined threshold may signify a maximum allowable percent deviation from a baseline amplitude, such as, e.g., e.g., 15, 16, 17, 18, 19, 20, 21, 22, 23, 24, 25 or more percent relative to a relative signal amplitude change, or any other percent deviation in a range of about, e.g., 10 to 40 percent. In another example, for a deviation in the time domain, the predetermined threshold may signify a maximum allowable percent deviation from a baseline deviation in time, such as, e.g., e.g., 1, 2, 3, 4, 5, 6, 7, 8, 9, 10 or more percent relative to a relative time-based change (e.g., based on sensor sampling rate such as 50 Hertz (Hz)), or any other percent deviation in a range of about, e.g., 0.1 to 20.0 percent. In another example, for a deviation in the frequency domain, the predetermined threshold may signify a maximum allowable percent deviation from a baseline deviation in frequency, such as, e.g., e.g., 1, 2, 3, 4, 5, 6, 7, 8, 9, 10 or more percent relative to a relative frequency-based change (e.g., based on a signal range such as between 0.015 and 0.4 Hz), or any other percent deviation in a range of about, e.g., 0.1 to 20.0 percent. In some embodiments, the predetermined threshold may be different for the deviation than the number of mismatches, or may be for a combination of the deviation and the number of mismatches (e.g., a weighted sum, a sum, or other function or any combination thereof). Where the PPG sensor 140 is defective, the testing fixture 110 may output to the user computing device, e.g., via the graphical user interface, an alert indicating that the PPG signal under test is defective.

In some embodiments, where the deviation does not exceed a predetermined threshold, the calibration engine 112 may determine offset or bias of the PPG sensor 140 that indicates a degree to which the PPG sensor 140 produces an output signal is above or below the expected output signal and/or illumination pattern according to the deviation. In some embodiments, the calibration engine 112 may reprogram the PPG sensor 140 to apply the offset to the measurements of the PPG sensor 140. As a result, the offset may compensate for the deviation in the measurements of the PPG sensor 140 to improve the accuracy of the PPG sensor 140.

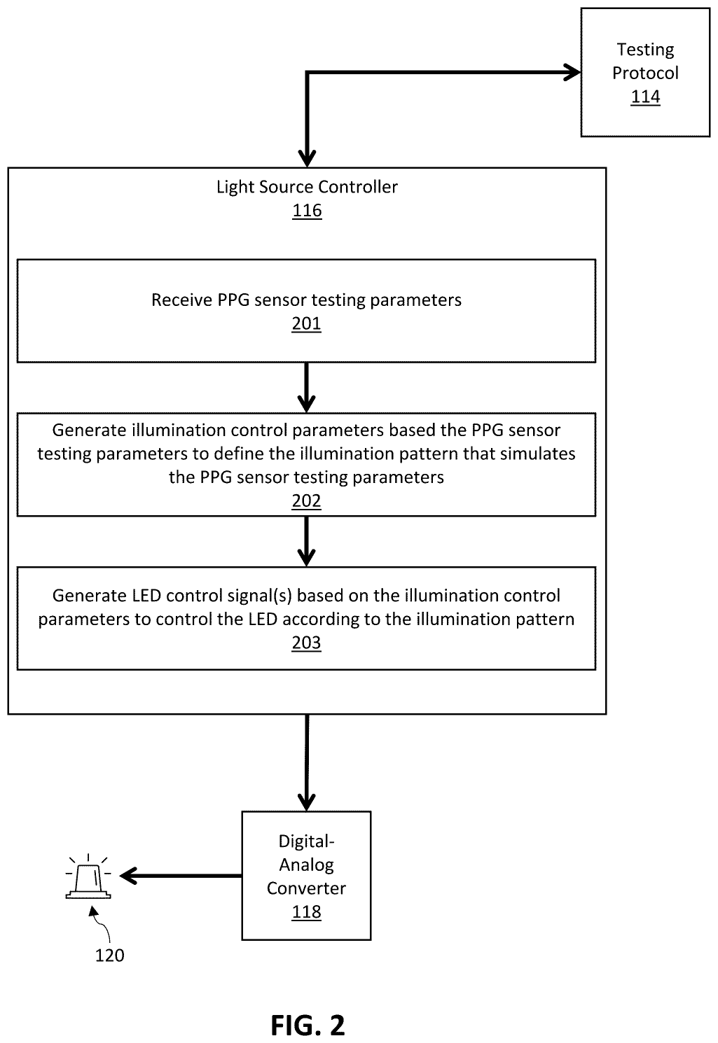

Referring now to FIG. 2, a light source controller of a testing fixture for automated testing of PPG sensor devices using a light source is illustrated in accordance with one or more embodiments of the present disclosure.

In some embodiments, the light source controller 116 may control the light source 120 based on a testing protocol 114 that represents a particular cardiac behavior represented by PPG sensor testing parameters. Accordingly, at block 201, the light source controller 116 may receive the PPG sensor testing parameters representing the cardiac behavior via the testing protocol 114, as detailed above.

In some embodiments, at block 202, the light source controller may generate light source control parameters that define an illumination pattern. In some embodiments, the illumination pattern simulates the PPG sensor testing parameters representing the cardiac behavior.

In some embodiments, at block 203, the light source controller 116 may generate one or more light source control signal(s) based on the actuation control parameters as detailed above to control the light source 120 according to the illumination pattern. The light source control signal(s) may include digital signals configured to cause the light source 120 to modulate intensity to each intensity level of a sequence of intensity levels defined by illumination pattern.

In some embodiments, the light source controller 116 outputs the light source control signals, via the DA converter 118, to the light source 120. As a result, the light source 120 is controlled to power the light source 120 to emit light towards the PPG sensor 140 at a sequence of intensity levels according to the illumination pattern to simulate the time-varying reflectivity of a subject's tissue when presenting the cardiac behavior.

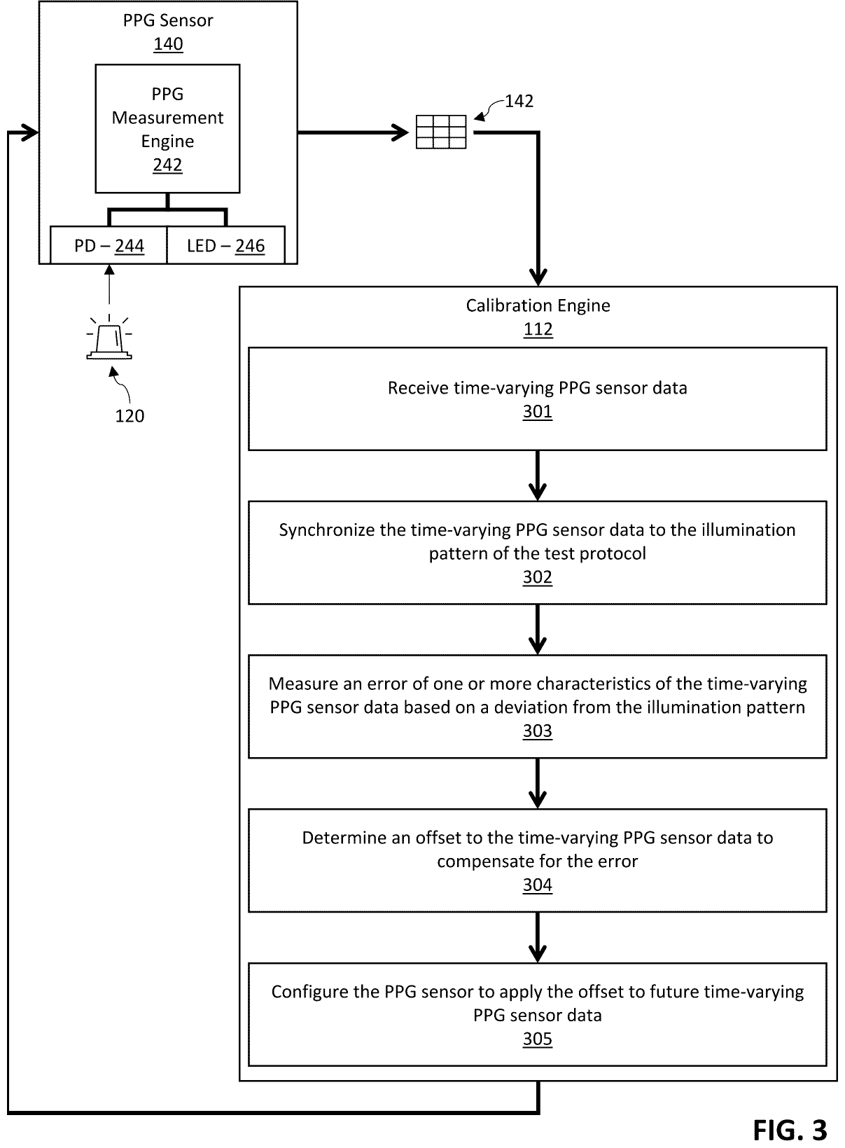

Referring now to FIG. 3, a calibration engine of a testing fixture for automated calibration of PPG sensor devices based on testing with a light source is illustrated in accordance with one or more embodiments of the present disclosure.

In some embodiments, the PPG sensor 140 may include a PPG measurement engine 242 configured to measure light detected on a subject's tissue as a way to measure cardiac activity. To do so, the PPG sensor 140 may include a light emitting diode (LED) 246 and a photodetector (PD) 244. In some embodiments, the PPG sensor 140 may employ the LED 246 to inject light into the subject's tissue and the PD 244 to receive light that reflects and/or scatters and exits the tissue. A PPG signal include time-varying PPG sensor data 142 may include the amplitude of the reflected and/or scattered light that is modulated with volumetric change in blood volume in the tissue. In some embodiments, the LED 246 may be a green LED, red LED, or an infrared (IR) LED. When more than one light emitter is used, the plurality can include the same or different light emitters (with different emission wavelengths). For example, a combination of one or more green LEDs and IR LEDs may be used. In some variations, the light emitting diodes emit light with a peak spectral response between about 400 and 620 nm.

In some embodiments, the light source 120 may directly produce light at an intensity that simulates the reflectivity of a subject's tissue for each stage in a cardiac cycle. Thus, the PD 244 may detect direct illumination from the light source 120 rather than a reflection of light from the LED 246. Thus, because the light source 120 directly provides light according to variations in intensity level corresponding to cardiac behaviors, the PD 244 may detect the light to produce an output signal including time-varying PPG sensor data 142 irrespective of light produced by the LED 246.

In some embodiments, at block 301, the calibration engine 112 may receive the time-varying PPG sensor data 142 of the PPG signal.

In some embodiments, at block 302, the calibration engine 112 may synchronize the time-varying PPG sensor data 142 to the illumination pattern of the test protocol 114. Thus, time in the time-varying PPG sensor data 142 may be aligned with time in the illumination pattern in order to directly compare the PPG measurements at each time point to the actuation control parameters of the illumination pattern at each time point.

In some embodiments, at block 303, the calibration engine 112 may measure an error in one or more characteristics of the time-varying PPG sensor data based on a deviation from the illumination pattern. The characteristic(s) may include, e.g., error in amplitude, error in duration, mismatch(es), among other sources of error or any combination thereof.

In some embodiments, at block 304, the calibration engine 112 may determine an offset to the time-varying PPG sensor data to compensate for the error.

In some embodiments, at block 305, the calibration engine 112 may configure the PPG sensor 140 to apply the offset to future time-varying PPG sensor data, thus compensating for the error in the time-varying PPG sensor data.

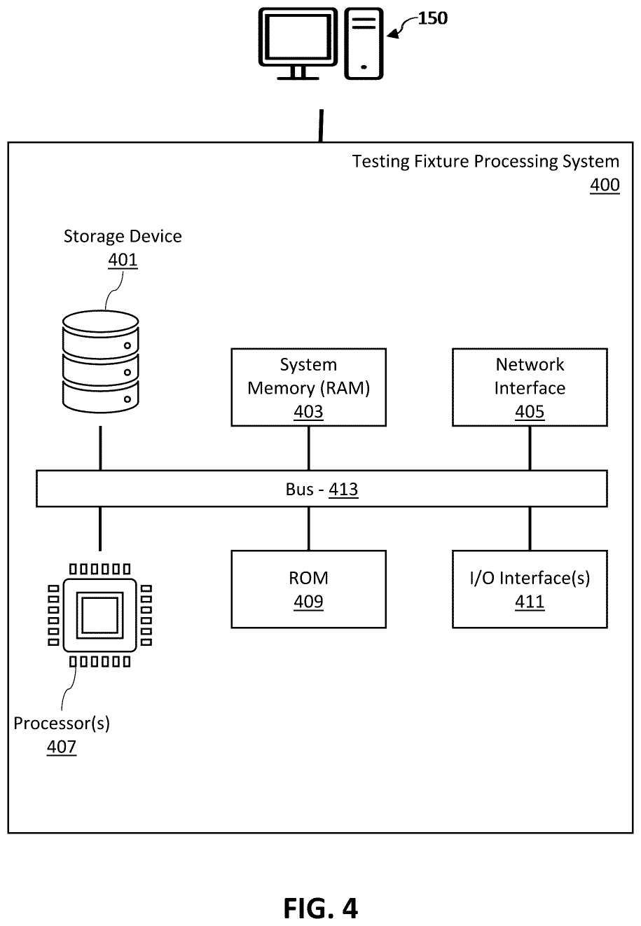

Referring now to FIG. 4, a processing system for a testing fixture for automated testing and calibration of PPG sensor devices is illustrated in accordance with one or more embodiments of the present disclosure.

The testing fixture processing system 400 may be a digital computer that, in terms of hardware architecture, generally includes a processor 409, input/output (I/O) interfaces 411, a network interface 405, a data store 401, and system memory (RAM) 403. It should be appreciated by those of ordinary skill in the art that FIG. 4 depicts the testing fixture processing system 400 in an oversimplified manner, and a practical embodiment may include additional components and suitably configured processing logic to support features described herein or known or conventional operating features that are not described in detail herein.

The components (409, 411, 405, 401, and 403) are communicatively coupled via a local interface 413. The local interface 413 may be, for example, but not limited to, one or more buses or other wired or wireless connections, as is known in the art. The local interface 413 may have additional elements, which are omitted for simplicity, such as controllers, buffers (caches), drivers, repeaters, and receivers, among many others, to enable communications. Further, the local interface 413 may include address, control, and/or data connections to enable appropriate communications among the aforementioned components.

The processor 409 is a hardware device for executing software instructions. The processor 409 may be any custom made or commercially available processor, a central processing unit (CPU), an auxiliary processor among several processors associated with the testing fixture processing system 400, a semiconductor-based microprocessor (in the form of a microchip or chip set), or generally any device for executing software instructions. When the testing fixture processing system 400 is in operation, the processor 409 is configured to execute software stored within the system memory (RAM) 403, to communicate data to and from the system memory (RAM) 403, and to generally control operations of the testing fixture processing system 400 pursuant to the software instructions. The I/O interfaces 411 may be used to receive user input from and/or for providing system output to one or more devices or components. User input may be provided via, for example, a keyboard, touchpad, and/or a mouse. System output may be provided via a display device and a printer (not shown). I/O interfaces 411 may include, for example, a serial port, a parallel port, a small computer system interface (SCSI), a serial ATA (SATA), a fiber channel, Infiniband, iSCSI, a PCI Express interface (PCI-x), an infrared (IR) interface, a radio frequency (RF) interface, and/or a universal serial bus (USB) interface.

The network interface 405 may be used to enable the testing fixture processing system 400 to communicate on a network, such as the cloud 12. The network interface 405 may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n/ac). The network interface 405 may include address, control, and/or data connections to enable appropriate communications on the network. A data store 401 may be used to store data. The data store 401 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 401 may incorporate electronic, magnetic, optical, and/or other types of storage media. In one example, the data store 401 may be located internal to the testing fixture processing system 400 such as, for example, an internal hard drive connected to the local interface 413 in the testing fixture processing system 400. Additionally, in another embodiment, the data store 401 may be located external to the testing fixture processing system 400 such as, for example, an external hard drive connected to the I/O interfaces 411 (e.g., SCSI or USB connection). In a further embodiment, the data store 401 may be connected to the testing fixture processing system 400 through a network, such as, for example, a network attached file server.

The system memory (RAM) 403 and/or the read only memory (ROM) 409 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the system memory (RAM) 403 may incorporate electronic, magnetic, optical, and/or other types of storage media. Note that the system memory (RAM) 403 may have a distributed architecture, where various components are situated remotely from one another but can be accessed by the processor 409. The software in system memory (RAM) 403 may include one or more software programs, each of which includes an ordered listing of executable instructions for implementing logical functions. The software in the system memory (RAM) 403 includes a suitable operating system (O/S) and one or more programs. The operating system controls the execution of other computer programs, such as the one or more programs, and provides scheduling, input-output control, file and data management, memory management, and communication control and related services. The one or more programs may be configured to implement the various processes, algorithms, methods, techniques, etc. described herein, such as related to the automated testing and/or calibration of the PPG sensor 140.

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTRO, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

The aforementioned examples are, of course, illustrative and not restrictive.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A testing fixture comprising:
a light source positioned so as to emit light towards a photoplethysmogram (PPG) sensor;
wherein each light source is configured to be modulated to vary an intensity level of the light;
a light source controller configured to:
receive a testing protocol comprising a plurality of PPG sensor testing parameters;
wherein the plurality of PPG sensor testing parameters is representative of a cardiac behavior of a subject;
generate an illumination pattern defined by the PPG sensor testing parameters;
wherein the illumination pattern comprises a mapping of the PPG sensor testing parameters to a sequence of intensity levels;
generate a light source control signal comprising a mapping of the sequence of intensity levels to a plurality of electrical energy modulations that, when communicated to the light source, is configured to modulate the light emitted by the light source to produce the sequence of intensity levels;
control the light source according to the illumination pattern to emit the light such that the light varies in intensity so as to present the sequence of intensity levels to the PPG sensor;
receive a PPG signal comprising time-varying PPG sensor data representative of measurements of the plurality of intensity levels;
determine that the PPG sensor is defective based at least in part on an error of one or more characteristics of the time-varying PPG sensor data relative to the illumination pattern; and
generate at least one alert to a user computing device to alert at least one user of that the PPG sensor is defective.

2. The testing fixture of claim 1, further comprising a calibration engine configured to:
receive the PPG signal comprising time-varying PPG sensor data representative of measurements of the plurality of intensity levels;
determine an offset to the time-varying PPG sensor data based at least in part on the error of the one or more characteristics; and
configure the PPG sensor to apply the offset to future time-varying PPG sensor data so as to compensate for the error.

3. The testing fixture of claim 1, wherein the light source comprises at least one light emitting diode (LED).

4. The testing fixture of claim 1, wherein the light source comprises at least one of:
at least one red LED,
at least one green LED,
at least one infrared (IR) LED, or
at least one ultraviolet (UV) LED.

5. The testing fixture of claim 1, wherein the light source is configured to emit the light at a plurality of intensity levels based on the light source control signal.

6. The testing fixture of claim 1, wherein the light source control signal comprises a digital signal having a predetermined bit depth.

7. The testing fixture of claim 6, further comprising a digital to analog converter configured to convert the light source control signal to an analog signal to power the light source according to the illumination pattern.

8. The testing fixture of claim 6, wherein the predetermined bit depth is associated with a number of intensity levels of the illumination pattern.

9. A method comprising:
receiving, by at least one processor of a testing fixture, a testing protocol comprising a plurality of PPG sensor testing parameters;
wherein the plurality of PPG sensor testing parameters is representative of a cardiac behavior of a subject;
wherein the testing fixture comprises:
a light source positioned so as to emit light towards a photoplethysmogram (PPG) sensor;
wherein each light source is configured to be modulated to vary an intensity level of the light;
generating, by the at least one processor, an illumination pattern defined by the PPG sensor testing parameters;
wherein the illumination pattern comprises a mapping of the PPG sensor testing parameters to a sequence of intensity levels;
generating, by the at least one processor, a light source control signal comprising a mapping of the sequence of intensity levels to a plurality of electrical energy modulations that, when communicated to the light source, is configured to modulate the light emitted by the light source to produce the sequence of intensity levels;
controlling, by the at least one processor, the light source according to the illumination pattern to emit the light such that the light varies in intensity so as to present the sequence of intensity levels to the PPG sensor;
receiving, by the at least one processor, a PPG signal comprising time-varying PPG sensor data representative of measurements of the plurality of intensity levels;
determining, by the at least one processor, that the PPG sensor is defective based at least in part on an error of one or more characteristics of the time-varying PPG sensor data relative to the illumination pattern; and
generating, by the at least one processor, at least one alert to a user computing device to alert at least one user of that the PPG sensor is defective.

10. The method of claim 9, further comprising a calibration engine configured to:
receiving, by the at least one processor, the PPG signal comprising time-varying PPG sensor data representative of measurements of the plurality of intensity levels;
determining, by the at least one processor, an offset to the time-varying PPG sensor data based at least in part on the error of the one or more characteristics; and
configuring, by the at least one processor, the PPG sensor to apply the offset to future time-varying PPG sensor data so as to compensate for the error.

11. The method of claim 9, wherein the light source comprises at least one light emitting diode (LED).

12. The method of claim 9, wherein the light source comprises at least one of:
at least one red LED,
at least one green LED,
at least one infrared (IR) LED, or
at least one ultraviolet (UV) LED.

13. The method of claim 9, wherein the light source is configured to emit the light at a plurality of intensity levels based on the light source control signal.

14. The method of claim 9, wherein the light source control signal comprises a digital signal having a predetermined bit depth.

15. The method of claim 14, further comprising converting, by the at least one processor, the light source control signal to an analog signal to power the light source according to the illumination pattern.

16. The method of claim 14, wherein the predetermined bit depth is associated with a number of intensity levels of the illumination pattern.

17. A testing fixture comprising:
a light source positioned so as to emit light towards a photoplethysmogram (PPG) sensor;
wherein each light source is configured to be modulated to vary an intensity level of the light;
a light source controller configured to:
receive a testing protocol comprising a plurality of PPG sensor testing parameters;
wherein the plurality of PPG sensor testing parameters is representative of a cardiac behavior of a subject;
generate an illumination pattern defined by the PPG sensor testing parameters;
wherein the illumination pattern comprises a mapping of the PPG sensor testing parameters to a sequence of intensity levels;
generate a light source control signal comprising a mapping of the sequence of intensity levels to a plurality of electrical energy modulations that, when communicated to the light source, is configured to modulate the light emitted by the light source to produce the sequence of intensity levels;
control the light source according to the illumination pattern to emit the light such that the light varies in intensity so as to present the sequence of intensity levels to the PPG sensor.

18. The testing fixture of claim 17, wherein the light source comprises at least one light emitting diode (LED).

19. The testing fixture of claim 17, wherein the light source comprises at least one of:
at least one red LED,
at least one green LED,
at least one infrared (IR) LED, or
at least one ultraviolet (UV) LED.

20. The testing fixture of claim 17, wherein the light source is configured to emit the light at a plurality of intensity levels based on the light source control signal.
